(19) United States
(12) Patent Application Publication
(10) Pub. No.: US 2009/0082716 A1
(43) Pub. Date: Mar. 26, 2009

(54) Linear to Torsional Converter for Phaco Handpieces

(76) Inventor: Takayuki Akahoshi Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/872,009

(22) Filed: Oct. 13, 2007

Related U.S. Application Data
(60) Provisional application No. 60/829,509, filed on Oct. 13, 2006.

(51) Int. Cl. A61F 9/007

(52) U.S. Cl. 604/22; 74/25

(57) ABSTRACT

A linear-to-torsional converter changes the linear motion imparted by a phaco handpiece to torsional or rotational motion. The converter attaches to the needle mount of a conventional linear handpiece. A lock can be used to defeat the torsional motion and restore linear motion without requiring the converter to be removed from the handpiece. Alternatively the converter can be built into a handpiece.

### PRIORITY

This application claims priority from U.S. patent application Ser. No. 60/829,509, filed Oct. 13, 2006, which is incorporated herein by reference.

### FIELD OF THE INVENTION

This disclosure relates to surgical instruments and surgical techniques used in eye surgery and more particularly, to phacoemulsification apparatus and methods for their use.

### BACKGROUND OF THE INVENTION

A common opthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle, commonly at ultrasonic frequencies, in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

Phacoemulsification handpieces typically provide vibrational movement in a longitudinal direction, that is, in a “back and forth” action along the central axis of the handpiece. Other phacoemulsification handpieces are constructed to provide torsional vibrational movement whereby the phacoemulsification needle is rotated back and forth through a selected arc. The term “linear” will be used throughout to identify movement of the phaco needle in the longitudinal direction, and the term “torsional” will be used throughout to identify back-and-forth rotational movement.

Phacoemulsification handpiece are well represented in the prior art. Needles and tips of varying configurations are well known. A particular shape for a tip or needle is often dictated by the type of handpiece with which the needle is to be used. U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published U.S. Patent Application No. 2003/0004455 (Kadziauskas) teaches and describes a bi-manual phaco needle using separate emulsification and aspiration needles inserted into the eye simultaneously during surgery.

U.S. Pat. No. 5,162,044 (Gahn et al) teaches and describes a phacoemulsification transducer with rotatable handle and is typical of a phacoemulsification handpiece using a piezoelectric transducer to create linear movement imparted to the phacoemulsification needle.

U.S. Pat. No. 5,413,556 (Whittingham) teaches and describes a phacoemulsification handpiece using a piezoelectric motor to create linear movement.

U.S. Pat. No. 6,077,285 (Boukhny) teaches and describes a torsional ultrasound handpiece configured to impart both linear and torsional motion to a phacoemulsification needle.

U.S. Pat. No. 6,402,769 (Boukhny) is a continuation in part of the '285 patent and further particularizes the frequencies at which the crystals providing both the torsional and linear motion are activated.

The introduction of the torsional handpiece such as those described in the Boukhny '285 and '769 patents is a relatively recent development. I have become aware that most surgeons already owning phacoemulsification handpieces that operate solely to producer linear motion prefer to continue using these handpieces for a variety of reasons. Such surgeons may from time to time experience the need to use a phacoemulsification handpiece capable of providing torsional movement. The aforementioned Buchkny patents allow the user to switch back and forth between the linear mode and the torsional mode.

I have determined that a need exists for a converter which will allow a surgeon to adapt a phacoemulsification handpiece capable only of linear motion to produce torsional motion when desired. Such a linear to torsional converter is considerably less expensive than a torsional or torsional plus linear handpiece and does not require the surgeon to readapt to a new instrument with new control systems, accessories and a new “feel” when being used.

In a first example of the present invention, a mechanical linear-to-torsional converter is attached to a phacoemulsification handpiece providing linear vibration when torsional movement is desired. Thereafter, the converter is removed to return the handpiece to its linear mode.

In another example, a mechanical lock is used to switch selectively between linear and torsional movement.

The converter is configured to attach easily to standard fittings on phacoemulsification handpieces and to be operated without modifying the mechanical or electrical operation of the handpiece.

While the following describes an example or examples of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the descriptions and examples presented herein, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE DRAWINGS

These and further aspects of the invention will be best understood by reference to the accompanying drawings wherein:

FIG. 1 is a prior art linear phacoemulsification handpiece;

FIG. 2 is a sectional view of a second prior art phacoemulsification handpiece providing both linear and torsional movement;

FIG. 3 is a lateral schematic sectional view of a linear-to-torsional converter;

FIG. 4 is a view along 4-4 of FIG. 3;

FIG. 5 is a lateral schematic sectional view of a second example of the present invention;

FIG. 6 is a view along 6-6 of FIG. 5;

FIG. 7 is a lateral view of the device shown in FIG. 5 illustrating an external lock in the unlocked position;

FIG. 8 is a lateral schematic sectional view of the example shown in FIG. 5 in the locked position;

FIG. 9 is a view along 9-9 of FIG. 8; and

FIG. 10 is a lateral view of the device shown in FIG. 8 with the external lock in the locked position;

### DETAILED DESCRIPTION OF THE DRAWINGS

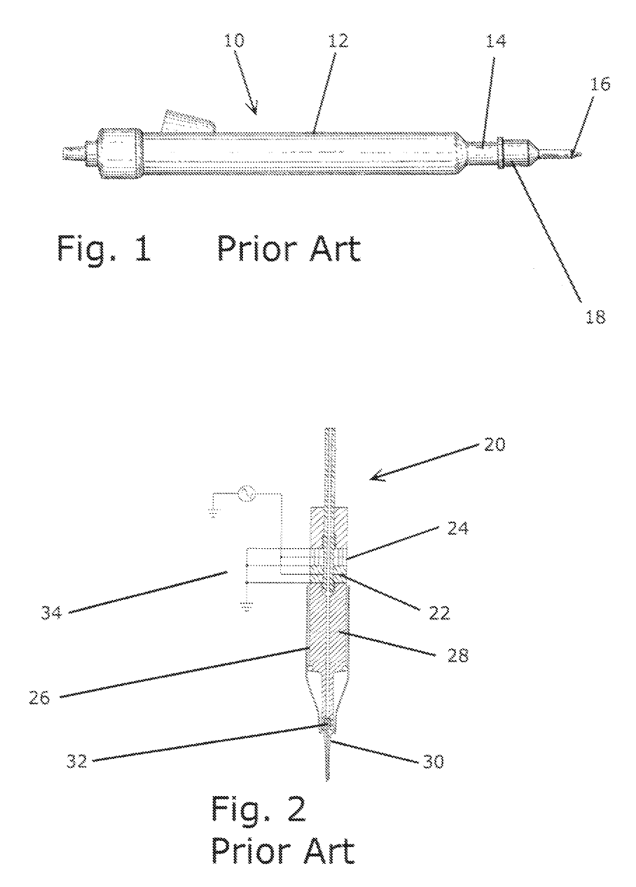

Referring now to FIG. 1, the numeral 10 identifies a prior art phacoemulsification handpiece as illustrated and described in U.S. Pat. No. 5,413,556. Handpiece 10 has a cylindrical body 12 within which the electrical and mechanical components that create linear vibratory movement are disposed. Body 12 terminates in a narrowed nose portion 14 to which a phacoemulsification needle 16 is threadably attached. Typically, phacoemulsification needle 16 has a mount at one end consisting of an externally threaded portion which is threaded into an internally threaded port on handpiece 10. In FIG. 1, an irrigation sleeve 18 is shown positioned over phacoemulsification needle 16.

Referring now to FIG. 2, the numeral 20 identifies a phacoemulsification handpiece capable of providing both linear and torsional vibration to a phacoemulsification needle. FIG. 2 is an illustration taken from U.S. Pat. No. 6,077,285 which also appears in U.S. Pat. No. 6,402,769.

A first set of piezoelectric crystals 22 and a second set of piezoelectric crystals 24 are disposed within housing 26 in contact with horn 28 to which a phacoemulsification needle 30 is threadably attached at mount 32. Crystals 22 are polarized to produce torsional motion while crystals 24 are polarized to produce linear motion. An electrical control system 34 is used to select the type and duration of the motion desired.

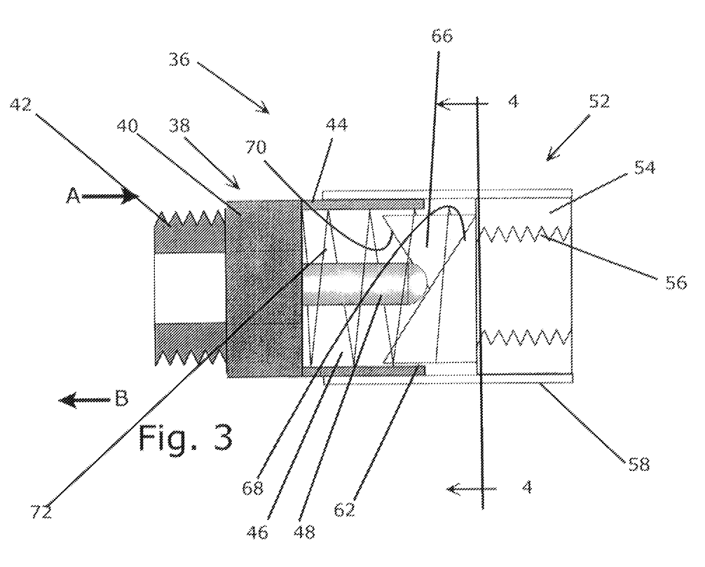

Referring now to FIG. 3, numeral 36 identifies a first example of a linear-to-torsional motion converter. Converter 36 comprises a handpiece mount 38 having a cylindrical base 40 with which a threaded plug 42 is integrally formed. Plug 42 is sized, shaped and threaded to allow it to be attached to a phacoemulsification handpiece at the mount at which a phacoemulsification needle would be attached.

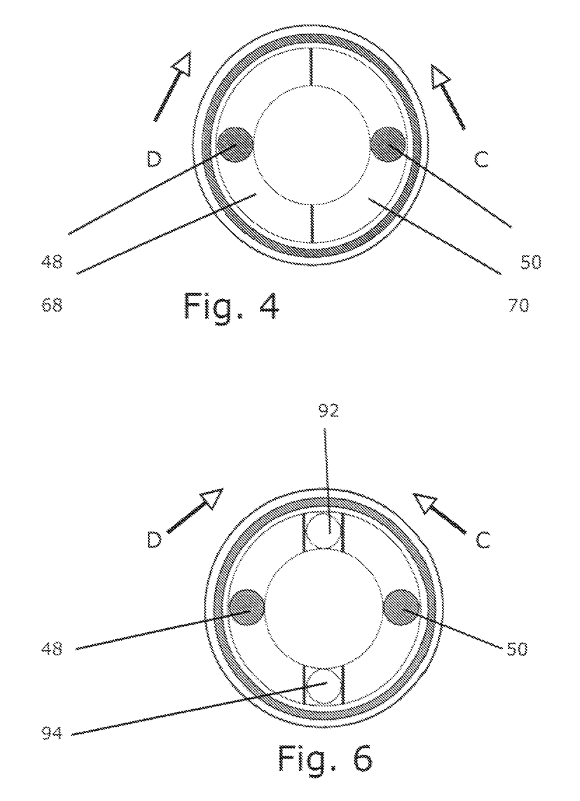

Base 40 has at its other end an upstanding cylindrical wall 44 forming and defining a first chamber 46 within which are disposed a pair of pins 48, 50 (best seen in FIG. 4).

Converter 36 also comprises a needle mount 52 having a base 54 within which an internally threaded passage 56 is formed, sized and shaped to threadably receive a phacoemulsification needle. Base 54 has an upstanding cylindrical wall 58 formed integrally therewith. A torsion block 62 is formed integral with base 54 comprising a first inclined block segment 64 and a second inclined block segment 66. Block segment 64 has a sloped outer face 68 while block segment 66 has an outer sloped face 70. In this example, the slope of first face 68 is opposite that of second face 70.

In this example, handpiece mount 38 fits slidably and rotatably within needle mount 52 such that needle mount 52 is free to rotate with respect to handpiece mount 38. A spring 90 is disposed within chamber 46 so as to oppose the rotation of needle mount 52 with respect to handpiece mount 38. That is, spring 90 opposes movement of needle mount 52 in direction D shown in FIG. 4.

Referring now to FIG. 4, it can be seen that pin 48 is positioned to contact sloped face 68 while pin 50 is positioned to contact sloped face 70. It can also be seen that, in this example, blocks 64, 66 are semicircular in shape.

Operation of converter 36 may now be described. The phacoemulsification handpiece to which mount 38 is attached provides a linear vibratory movement back and forth alternately in direction A and B. Moving in direction A, pin 48 contacts sloped face 68 and pin 50 contacts sloped face 70. When pin 48 pushes face 68 it imparts a torsional or twisting motion to needle mount 52 with respect to handpiece mount 38, while, at the same time, stressing spring 32 in direction C.

During that portion of the vibratory cycle where handpiece mount 38 is moving in direction B, spring 72 returns toward its unstressed position thereby pulling needle mount 52 in direction D. As this repetitive motion is continuously provided, needle mount 52 twists in both directions C and D providing a torsional motion to a phacoemulsification needle mounted to needle mount 52.

When linear motion only is desired, converter 36 can be unthreaded from the handpiece and the phacoemulsification needle can be threaded into the mount to which the converter had been previously attached.

In another example, a mechanical lock is provided which, when engaged, prevents needle mount 52 from moving linearly with respect to handpiece mount 38 and which further prevents any movement in directions C or D, thus returning its handpiece to its linear-only mode.

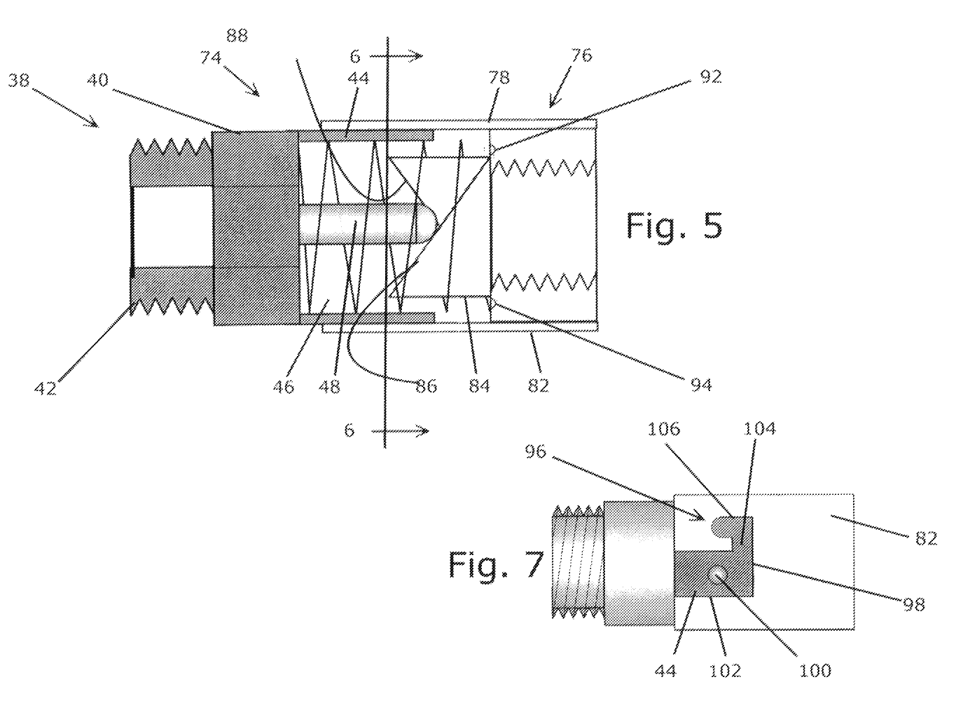

Referring now to FIG. 5, the numeral 74 identifies another example of a liner-to-torsional motion converter incorporating a locking system to allow converter 74 to impart torsional movement to a phacoemulsification needle when unlocked and linear movement when locked.

As described hereinabove, converter 74 comprises a handpiece mount 38 having a cylindrical base 40 with which a threaded plug 42 is integrally formed. Plug 42 is sized, shaped and threaded to allow it to be attached to a phacoemulsification handpiece at the mount at which a phacoemulsification needle would be attached.

Base 40 has at its other end an upstanding cylindrical wall 44 forming and defining a first chamber 46 within which are disposed a pair of pins 48, 50 as seen in FIG. 6.

Converter 74 also comprises a needle mount 76 having a base 78 within which an internally threaded passage 80 is formed, sized and shaped to threadably receive a phacoemulsification needle. Base 78 has an upstanding cylindrical wall 82 formed integrally therewith. A torsion block 84 is formed integral with base 78 and comprises a first inclined block face 86 and a second inclined block face 88. In this example, the slope of face 86 is opposite that of face 88.

In this example, handpiece mount 38 fits rotatably within needle mount 76 such that needle mount 76 is free to rotate with respect to handpiece mount 38 in directions C and D as shown in FIG. 6. A spring 90 is disposed within chamber 46 and is attached to block 84 so as to oppose the rotation of needle mount 76 with respect to handpiece mount 38. That is, spring 90 opposes movement of needle mount 76 in direction C as shown in FIG. 6. Spring 90 also opposes the outward linear motion of needle mount 76 with respect to handpiece mount 38.

Referring now to FIG. 6, it can be seen that pin 48 is positioned to contact face 86 while pin 50 is positioned to contact face 88.

Referring now to FIGS. 5 and 6, a pair of pin detents 92, 94 are formed in needle mount 76, positioned to communicate with chamber 46. Detents 92, 94 are also positioned to engage pins 48, 50 respectively when needle mount 76 is rotated through an arc of about 90° with respect to handpiece mount 38. To effect such rotation and to retain needle mount 76 in its rotated position a pin-and-groove lock 96 is provided, as seen in FIG. 7. Lock 96 comprises a locking slot 98 formed in wall 82 and a locking pin 100 formed on the outer surface of wall 44. Lock 96 is shown in its unlocked position in FIG. 7. “Unlocked” identifies that configuration of converter 74 in which needle mount 76 is free to rotate with respect to handpiece mount 38. Locking slot 98 is generally L-shaped and has a first, axial segment 102 communicating with a transverse segment 104 which, in turn, communicates with an axial retainer segment 106.

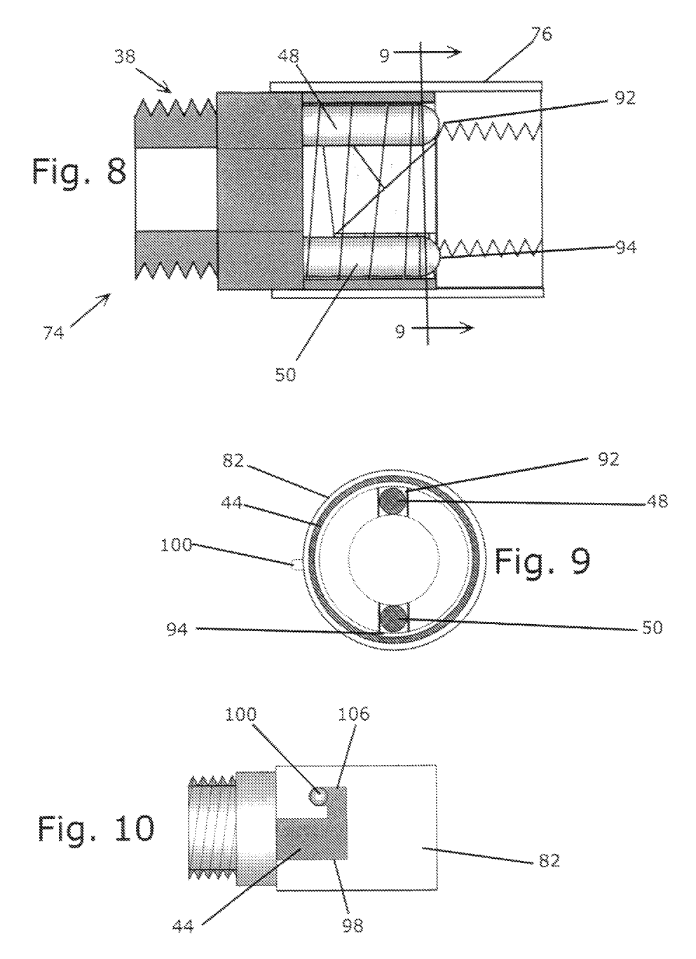

Referring now to FIG. 8, converter 74 is shown in its “locked” position, meaning that needle mount 76 is constrained from rotating with respect to handpiece mount 38. Needle mount 76 has been rotated to align pin 48 with detent 92 and pin 50 with detent 94. Preventing rotation of needle mount 76. As seen in both FIGS. 9 and 10, pin 100 is positioned in retainer segment 106 of slot 98 and protrudes from wall 82.

In this position, the linear motion created by the handpiece is transmitted to an attached needle in the linear direction only. To impart a torsional motion to the needle, needle mount 76 is rotated to disengage pin 100 from retainer segment 106, allowing needle mount 76 once again to rotate with respect to handpiece mount 38 and create a torsional movement as described hereinabove.

The examples described herein can be manufactured from titanium, a material that is light, corrosion-resistant, and of sufficient strength and stiffness to handle the vibratory stress created by a phacoemulsification handpiece. Converters constructed in accordance with the foregoing examples are expected to be about 10 mm in length and about 6 mm in diameter.

One manner in which the examples described herein find particular utility is the ability to create torsional motion with a linear-only handpiece which costs approximately one-half as much as a torsional handpiece and which does not need the extremely expensive control console that the handpiece described in FIG. 2 requires in order to control the changes between linear and torsional motion.

Another manner in which the above-described converter may be used is to manufacture a handpiece with the converter incorporated therein as a less expensive alternative than the handpieces described above.

I claim:

1. Apparatus for converting linear vibratory motion created by a phacoemulsification handpiece to torsional or rotational motion, said handpiece of the type having a needle mount to which a phacoemulsification needle is mountable, said apparatus comprising:
a first housing,
said first housing having a housing mount adapted to attach to said handpiece at said needle mount,
said first housing having at least one pin mounted therein extending in the direction said linear motion occurs;
a second housing sized and shaped to slidable and rotatably receive said first housing,
said second housing having a torsion block formed integrally and internally therewith,
said torsion block having at least one block segment inclined at a selected angle with respect to said pin and positioned to contact said pin when said first and second housings move slidably with respect to each other,
said block and, thereby, said second housing rotating in a first direction with respect to said first housing when said pin contacts said inclined block segment.

2. The apparatus as recited in claim 1 further comprising means formed on said second housing to mount a phacoemulsification needle thereon.

3. The apparatus as recited in claim 1 wherein said first housing has first and second pins mounted therein.

4. The apparatus as recited in claim 3 wherein said second housing has first and second inclined block segments mounted therein,
each said inclined block segment positioned to contact one said pin when said first and second housings move one toward the other.

5. The apparatus as recited in claim 4 wherein said first and second pins are positioned diametrically opposite one another and wherein said first inclined block segment is inclined at an angle opposite that of said second inclined block segment.

6. The apparatus as recited in claim 1 wherein said apparatus further comprises means disposed within said converter to urge said housing to rotate in a second direction opposite that of said first direction.

7. The apparatus as recited in claim 6 wherein said urging means comprises a spring.

8. The apparatus as recited in claim 1 wherein said apparatus further comprises means to lock said first and second housings together whereby said second housing does not rotate.

9. Apparatus for switching between linear vibratory motion and torsional or rotational motion in a handpiece providing linear motion only, said apparatus comprising:
a first housing,
said first housing having at least one pin mounted therein extending in the direction said linear motion occurs;
a second housing sized and shaped to slidable and rotatably receive said first housing,
said second housing having a torsion block formed integrally and internally therewith,
said torsion block having at least one block segment inclined at a selected angle with respect to said pin and positioned to contact said pin when said first and second housings move slidably with respect to each other,
said block and, thereby, said second housing rotating in a first direction with respect to said first housing when said pin contacts said inclined block segment.

10. The apparatus as recited in claim 9 further comprising means formed on said second housing to mount a phacoemulsification needle thereon.

11. The apparatus as recited in claim 9 wherein said first housing has first and second pins mounted therein.

12. The apparatus as recited in claim 4 wherein said first and second pins are positioned diametrically opposite one another and wherein said first inclined block segment is inclined at an angle opposite that of said second inclined block segment.

13. The apparatus as recited in claim 9 wherein said apparatus further comprises means disposed within said converter to urge said housing to rotate in a second direction opposite that of said first direction.

14. The apparatus as recited in claim 13 wherein said urging means comprises a spring.

15. The apparatus as recited in claim 9 wherein said apparatus further comprises means to lock said first and second housings together whereby said second housing does not rotate.

16. The apparatus as recited in claim 11 wherein said second housing has first and second inclined block segments mounted therein,
each said inclined block segment positioned to contact one said pin when said first and second housings move one toward the other.
